(19) United States
(12) Patent Application Publication
(10) Pub. No.: US 2021/0251963 A1
(43) Pub. Date: Aug. 19, 2021

(54) UREA DERIVATIVES FOR USE IN THE TREATMENT OF SUBJECTS WITH ELEVATED EXPRESSION AND/OR ACTIVITY OF SRPK1

(71) Applicant: Scandion Oncology A/S, Copenhagen Ø (DK)

(72) Inventors: Nils Aage Brünner, Hellerup (DK); Jan Stenvang, Copenhagen K (DK)

(73) Assignee: Scandion Oncology A/S, Copenhagen Ø (DK)

(21) Appl. No.: 17/272,808

(22) PCT Filed: Sep. 6, 2019
(86) PCT No.: PCT/EP2019/073796

§ 371 Date: Mar. 2, 2021

(30) Foreign Application Priority Data
Sep. 6, 2018 (EP) 18193008.2

(51) Int. Cl. A61K 31/41; A61K 31/573; A61K 31/565; A61K 31/138; A61K 45/06; A61K 31/4745; A61K 31/704; A61K 31/337; A61P 35/00

(52) U.S. Cl. CPC A61K 31/41; A61K 31/573; A61K 31/565; A61K 31/138; A61P 35/00; A61K 31/4745; A61K 31/704; A61K 31/337; A61K 45/06

(57) ABSTRACT

The present invention relates to treatment of diseases characterized by elevated expression and/or activity of SRPK1 with specific SRPK1 inhibitors and to methods for identifying subjects who may benefit from such treatment.

### TECHNICAL FIELD

The present invention relates to treatment of diseases characterized by elevated expression and/or activity of SRPK1 with specific SRPK1 inhibitors and to methods for identifying subjects which may benefit from such treatment.

### BACKGROUND

Protein kinases, being key regulators of most cellular pathways, are frequently associated with diseases, either as causative agents or as therapeutic intervention points.

A particular kinase, the serine/arginine-rich protein-specific kinase 1 (SRPK1) phosphorylates proteins involved in the regulation of several mRNA processing pathways including alternative splicing. SRPK1 has been reported to be overexpressed in multiple cancers including prostate, breast, lung and glioma (Oncotarget. 2017, 37, 61944). For example in breast cancer overexpression of SRPK1 has been found to correlate with the development and progression of breast cancer and possibly resistance to taxanes (Oncotarget, 2017, 8, 103327). Several studies have further identified that inhibition/down-regulation of SRPK1 results in tumor-suppressive effects, such as reduced angiogenesis and reduced cancer cell migration, thus identifying SRPK1 as a potential novel anti-cancer target.

SRPK1 has recently been reported to be overexpressed in multiple cancers, including prostate cancer, breast cancer, lung cancer, and glioma. Several studies have further shown that inhibition of SRPK1 has anti-cancer effects, and SRPK1 has therefore become a new candidate for targeted therapies. A recent report adds to this puzzle, showing that the main effect of SRPK1 overexpression in non-small-cell lung carcinoma is to stimulate a cancer stem cell-like phenotype. This pleiotropy might be related to preferential activation of different downstream signalling pathways by SRPK1 in various cancers.

A synthetic small-molecule SRPK1 inhibitor, SPHINX has been shown to be capable of inhibiting tumor cell growth in several cancers characterised by elevated SRPK1 expression, including non-small cell lung cancer and prostate cancer (Lui et al. 2016, Mavrou et al. 2015).

It is commonly known that most small-molecule kinase inhibitors interact with multiple members of the protein kinase family and may therefore result in unacceptable treatment-induced side effects. Hence, achieving selective inhibition of specific protein kinases is challenging but may be necessary for successful development of kinase inhibitors as treatment of human diseases (Nature Reviews Drug Discovery volume 11, page 21 (2012)). This is even more important when a kinase inhibitor is to be administered together with a cytotoxic drug as an additive effect on adverse events has been observed (Regorafinib) leading to a reduction in the dose of the cytotoxic drug.

Hence, there is a need in the art for developing selective SPRK1 inhibitors for improved treatment of diseases regulated by SRPK1, such as cancer.

### SUMMARY

The present inventors have surprisingly found that SCO-101 selectively inhibits SRPK1 and since it is a safe drug with very limited toxicity, SCO-101 and related compounds are considered useful for treatment of diseases characterised by overexpression and/or elevated activity of SRPK1.

The present disclosure thus provides selective SRPK1 inhibitors in the form of SCO-101 and related compounds useful for the treatment of subjects suffering from diseases being regulated by the kinase SRPK1; particularly subjects characterized by elevated expression and/or elevated activity of SRPK1.

### DESCRIPTION OF DRAWINGS

FIG. 1. Determination of IC50 towards SRPK1 for SCO-101.

FIG. 2. Response to SN38 in HT29 parental and SN38 resistant colon cancer cells, respectively.

FIG. 3. Effect of SCO-101 and SN38 in SN38 resistant HT29 colon cancer cells.

FIG. 4. Response to SN38, Srpkin340 and Sphinx31 upon single administration or in combined treatment in HT29 SN38 resistant colon cancer cells.

### DETAILED DESCRIPTION

The present inventors have surprisingly found that SCO-101 is a highly selective SRPK1 inhibitor with very limited side effects when administered orally to human beings. Hence, SCO-101 and related compounds constitute a novel treatment of diseases regulated by SRPK1 and particularly of subjects having elevated expression and/or activity of SRPK1.

A selective inhibitor of SRPK1 is intended to mean a compound which is capable of inhibiting the mean activity of SRPK1 to less than 10% of control, preferably to less than 8%, such as less than 7%, such as less than 6%, such as less than 5% of control. The selective SRPK1 inhibitor of the present disclosure may have some capability to inhibit other kinases, but preferably does not inhibit the activity of any other kinases to more than 20% of control, more preferably no more than 25% of control, even more preferably to more than 30% of control.

It is an aspect of the present disclosure to provide a method of treating a disease comprising administering to a subject an effective amount of a composition comprising a SRPK1 inhibitor of formula I,

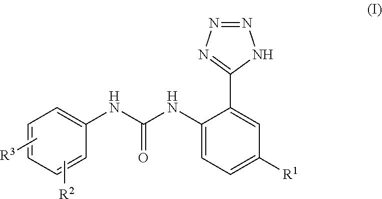

or a pharmaceutically acceptable salt thereof,

wherein

R¹, R² and R³ independently of each other represent hydrogen, halo, trifluoromethyl, nitro, alkyl, alkylcarbonyl, —NR^{a}R^{b}, —NR^{a}—CO—R^{b}, phenyl or heteroaryl; which phenyl is optionally substituted with halo, trifluoromethyl, nitro, —CO—NHR^{c}, —CO—

O—R^{c} or —CO—NR′R″;

wherein R^{c} is hydrogen, alkyl, or phenyl;

R′ and R″ independently of each other are hydrogen or alkyl; or

R′ and R″ together with the nitrogen to which they are attached form a 5- to 7-membered heterocyclic ring, which ring may optionally comprise as a ring member, one oxygen atom, and/or one additional nitrogen atom, and/or one carbon-carbon double bond, and/or one carbon-nitrogen double bond;

and which heterocyclic ring may optionally be substituted with alkyl;

R^{a} and R^{b} independently of each other are hydrogen or alkyl;

wherein the subject is characterized by an elevated expression and/or activity of Serine Arginine Protein Kinase 1 (SRPK1).

In one embodiment, the disease is a cancer.

In one embodiment, the SRPK1 inhibitor according to the present disclosure is of formula (II)

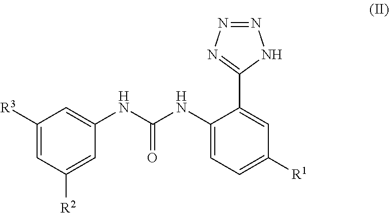

or a pharmaceutically acceptable salt thereof, wherein R¹, R² and R³ are as defined for formula (I).

In a particular embodiment of the present disclosure, the SRPK1 inhibitor of formula I is SCO-101, also known as NS3728 and Endovion:

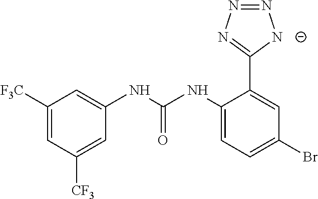 [SMILES: O=C(Nc1cc(C(F)(F)F)cc(C(F)(F)F)c1)Nc1ccc(Br)cc1-c1nnn[nH]1]

or a pharmaceutically acceptable salt thereof.

It is further an aspect of the present disclosure to provide a method of selecting a subject for treatment with the composition according to the present disclosure, said method comprising:
a. providing a sample comprising diseased tissue or diseased cells from the subject,
b. determining the expression level and/or activity of SRPK1 in said sample,
c. comparing said expression level and/or activity of SRPK1 with the expression level and/or activity of SRPK1 in a control sample,

wherein an expression level and/or activity of SRPK1 in the sample above the expression level in the control sample indicates that the subject is responsive to treatment with an SRPK1 inhibitor as defined herein.

It is an aspect of the present disclosure to provide a method for treatment of a disease characterized by an elevated expression and/or activity of Serine Arginine Protein Kinase 1 (SRPK1) comprising administering to a subject an effective amount of a composition comprising the SRPK1 inhibitor as defined herein and optionally a further medicament, wherein sample comprising diseased tissue or diseased cells obtained from said subject comprises an elevated expression and/or activity of SPRK1.

It is a further aspect of the present disclosure to provide a composition comprising an effective amount of a SRPK1 inhibitor as defined herein for use in the treatment of a disease characterized by an elevated expression and/or activity of Serine Arginine Protein Kinase 1 (SRPK1).

Further, one embodiment of the present disclosure relates to use of a SRPK1 inhibitor as defined herein for the manufacture of a medicament for treatment of a disease characterized by an elevated expression and/or activity of Serine Arginine Protein Kinase 1 (SRPK1).

SRPK1

SRPK1 is an intracellular kinase, which belongs to the serine/threonine kinase subfamily. It exists in three isoforms: SRPK1, SRPK 2 and SRPK3, which have different cellular distribution.

The SRPK1 gene encodes a serine/arginine protein kinase, which is specific for the phosphorylation of the SR (serine/arginine rich domain) family of splicing factors, which contains more than 100 members. Thus, it is a major regulator of splicing factors. The SR family of splicing factors are a part of the mechanism that prevents exon skipping in precursor mRNA, thus ensuring the accuracy of the exon/intron splicing. The splicing factors are also involved in the alternative splicing process, thus it is a part of both the normal and alternative splicing of precursor mRNA. The SR proteins consist of one or two N-terminal RNA recognition motif domain (RRM domain) and a C-terminal domain rich in the amino acids serine and arginine (the SR domain). The SR domain in the SR proteins is recognized and phosphorylated at serine residues by the SRPK1 at multiple sites. SRPK1 recognizes serine residues in serine-arginine or arginine-serine dipeptide motifs e.g. RSRSRS. The SRPK1 mediated phosphorylation plays an important role in the transport of shuttling SR proteins from the cytoplasm into the nucleus.

SRPK1 phosphorylates the SR domain at approximately 12 serine residues. It phosphorylates its substrate in a C-terminal to N-terminal direction using a dual-track mechanism, with both processive phosphorylation steps, where the kinase stays attached to the substrate after each round of phosphorylation, and distributive phosphorylation steps, where the kinase dissociates from the substrate after each round of phosphorylation. The first approximately 1-8 steps proceeds in a processive way, and the last approximately 9-12 steps is in a distributive way. During the last steps, mechanical stress signals the substrate to dissociate from the SRPK1. The SRPK1 gene is located in the human chromosome 6 on the reverse strand. The coding region of the gene is positioned from 35.832.966-35.921.342 bp, with the size of 88.376 bp. It consists of 16 exons, which are highly conserved among vertebrates.

SRPK1 is expressed in all non-pathologic tissues in approximately equal amounts. It is an intracellular protein kinase that is located both in the cytoplasm and the nucleus and has been shown to be involved in mRNA maturation, chromatin regulation and mitosis. In the cytoplasm, the SRPK1 is bound in a complex with chaperones from which it can be released by a change in the complex due to upstream signals, such as stress signals and cell cycle dependent signals. Once released from the complex, SRPK1 has been shown to be imported to the nucleus. In the nucleus, it can phosphorylate non-shuttling SR proteins.

SRPK1 consists of a highly conserved protein kinase domain that is separated in two halves by a spacer sequence. The N-terminal part of the kinase domain encodes the smaller loop. It is composed mostly of β-strands and contains the ATP binding site. The C-terminal part of the kinase domain encodes the larger loop. It is composed mostly of α-helixes and contains the substrate binding site. The SR domain in the substrate first binds to the large loop of the kinase domain, which induces a confirmation change that allows for the substrates RRM domain to bind to the kinase which initiates the phosphorylation. The spacer sequence does not affect the activity of the kinase. In addition to the spacer sequence, SRPK1 also contains a non-conserved N-terminal extension, which is not necessary for the kinase activity.

The kinase activity of SRPK1 is thought to be constitutively active. The structure of the activation loop is rather short and lacks a regulatory phosphorylation site. Thus, the activation loop adopts a stable conformation that permits substrates to access the active site continuously. Studies have shown that alternative residues can re-establish interactions that are lost upon mutations of some residues in the active site, thus making the SRPK1 resilient to inactivation. Even though the spacer and N-terminal extension are not required for the kinase activity, they are important for the localization and regulation of SRPK1. The N-terminal can either enhance the catalytic activity through phosphorylation from CK2 (casein kinase 2), or suppress the activity by the binding of nuclear scaffold proteins. The spacer sequence is the regulator of the intracellular location of SRPK1. In the absence of the spacer, the distribution pattern of SRPK1 changes from being mainly in the cytoplasm to exclusively being in the nucleus. The spacer sequence is predicted to lack a secondary structure, and most likely be unfolded. This provides an interaction site for members of the chaperone family in the cytoplasm.

Subject Selection

It is an aspect of the present disclosure to provide a method of selecting a subject for treatment with the SRPK1 inhibitor according to the present disclosure, said method comprising:
a. providing a sample comprising diseased tissue or diseased cells from the subject,
b. determining the expression level and/or activity of SRPK1 in said sample, and
c. comparing said expression level and/or activity of SRPK1 with the expression level and/or activity of SRPK1 in a control sample,

wherein an expression level and/or activity of SRPK1 in the sample above the expression level in the control sample indicates that the subject is responsive to treatment with the SRPK1 inhibitor as defined herein.

The subject may then be treated with an SRPK1 inhibitor as described elsewhere herein.

Determining Expression and/or Activity of BCRP

The human breast cancer resistance protein (BCRP, gene symbol ABCG2) is an ATP-binding cassette (ABC) efflux transporter and has been found to confer resistance to certain chemotherapeutic agents, such as irinotecan, SN38, mitoxantrone and topotecan.

In one embodiment, the method of selecting a subject for treatment with the SRPK1 inhibitor according to the present disclosure further comprises determining the expression level and/or activity of BCRP in said sample; and comparing said expression level and/or activity of BCRP with the expression level and/or activity of BCRP in a control sample; wherein an expression level and/or activity of BCRP above the expression level and/or activity of BCRP in the control sample indicates that the subject is responsive to treatment with the SRPK1 inhibitor as defined herein.

Cancer

In some aspects, the present disclosure relates to the treatment of cancer in subjects with elevated expression levels and/or activity of SRPK1. In some aspects, the present disclosure relates to the treatment of cancer in subjects with elevated activation (over activation) of SRPK1.

In one embodiment, the cancer is resistant to treatment with an anti-cancer agent. If a cancer is resistant, co-treatment with a SRPK1 inhibitor is capable of re-sensitising the cancer to anti-cancer agent in question. Resistance of cancers may be either de novo resistance or acquired resistance. In general, a cancer is regarded as resistant to a particular anti-cancer agent if a patient treated with the clinically accepted dosage of the anti-cancer agent does not respond as expected to the anti-cancer agent, i.e. in case of worsening, growth, or spread of the cancer (progressive disease). Whether a cancer is drug-sensitive or -resistant can be determined by the skilled person.

In one embodiment, the cancer to be treated according to the present disclosure may be selected from the group consisting of lung cancer (non small cell lung cancer and small cell lung cancer), Glioblastomas, Head and neck cancers, Malignant melanomas, Basal cell skin cancer, Squamous cell skin cancer, Breast cancer, Liver cancer, Pancreatic cancer, Prostate cancer, Colorectal cancer, anal cancer, Cervix uteri cancer, Bladder cancer, Corpus uteri cancer, Ovarian cancer, Gall bladder cancer, Sarcomas, Leukemia's (myeloid and lymphatic), Lymphomas, Myelomatosis. In some embodiments, the cancer is selected from the group consisting of colon cancer, breast cancer, prostate cancer, pancreatic cancer, brain cancer, ovarian cancer skin cancer, gastrointestinal cancer and lung cancer.

In one embodiment, the cancer is resistant to an anti-cancer agent, such as a chemotherapeutic agent. In one embodiment, the resistance is de novo resistance. In one embodiment, the resistance is acquired resistance.

In one embodiment, the cancer is metastatic.

In one embodiment, the cancer is colorectal cancer.

In one embodiment. the cancer is metastatic colorectal cancer.

In one embodiment, the cancer is breast cancer.

In one embodiment, the cancer is metastatic breast cancer.

In one embodiment, the cancer is pancreatic cancer.

In one embodiment, the cancer is brain cancer.

In one embodiment, the cancer is ovarian cancer.

In one embodiment, the cancer is skin cancer.

In one embodiment, the cancer is gastrointestinal cancer.

In one embodiment, the cancer is glioblastoma.

In one embodiment, the cancer is a solid tumour such as a solid tumour selected from sarcoma, carcinoma and lymphoma.

In one embodiment, the cancer is not a solid tumour. For example, the cancer may be a hematological malignancy including but not limited to leukemias and lymphomas.

In one embodiment, the cancer is prostate cancer, such as metastatic prostate cancer.

In one embodiment, the cancer is a steroid hormone receptor positive and steroid hormone sensitive cancer, e.g. an estrogen receptor positive cancer, a progesterone receptor positive cancer or an androgen receptor positive cancer.

In one embodiment, the cancer is resistant to anti-hormonal treatment.

Combination Treatment

In one embodiment of the present disclosure, the SRPK1 inhibitor is administered with a further medicament, e.g. an anti-cancer agent or is combined with another treatment modality such as radiation therapy. In other embodiments, the SRPK1 inhibitor is used in mono-therapy.

In some embodiments, the SRPK1 inhibitor as disclosed herein is administered before, during and/or after the subject has received treatment with a further medicament, optionally wherein the treatment by the further medicament has not been effective. In one embodiment, the treatment as described herein is additive. In one embodiment, the treatment as described herein is synergistic. In one embodiment, the SRPK1 inhibitor potentiates the therapeutic effect of the further medicament.

The combination treatment according to the present disclosure may be treatment with one or more anti-cancer agents and/or radiation therapy. In a preferred embodiment, the combination treatment encompasses treatment with a chemotherapeutic agent or an anti-hormonal treatment (endocrine treatment) or an anti-angiogenic drug or an anti-metastatic drug. In one embodiment, the further medicament is an anti-cancer agent.

In some embodiments, the further medicament is a chemotherapeutic agent selected from the group consisting of topoisomerase inhibitors, anti-hormone agents, alkylating agents, mitotic inhibitors, antimetabolites, anti-tumour antibiotics, corticosteroids, targeted anti-cancer agents, differentiating agents and immunotherapy.

Chemotherapy drugs can be divided into groups based on factors such as how they work, their chemical structure, and their relationship to other drugs. Some drugs act in more than one way, and may belong to more than one group. In one embodiment the anti-cancer treatment of the present invention encompasses treatment with a SRPK-1 inhibitor in combination with more than one chemotherapeutic agent.

In one embodiment, the anti-cancer agent is a chemotherapeutic agent selected from the group consisting of a cytotoxic agent, a cytostatic agent, an anti-hormone agent, an anti-angiogenic agent, an immune-oncology agent and an anti-cancer biologic agent, e.g. antibody with a well-defined target.

In one embodiment, the chemotherapeutic agent is a cytotoxic agent or a cytostatic agent.

In a preferred embodiment, the anti-cancer agent is a chemotherapeutic agent and the SRPK1 inhibitor is co-administered with the chemotherapeutic agent.

The SRPK1 inhibitor may be administered prior to, simultaneously with and/or after the anti-cancer agent. In one embodiment the SRPK1 inhibitor is administered prior to the anti-cancer agent. In one embodiment the SRPK1 inhibitor is administered simultaneously with the anti-cancer agent, and in one embodiment, the SRPK1 inhibitor is administered before, simultaneously with, and after the anti-cancer agent.

Co-administration as used herein refers to administration of a SRPK1 inhibitor and an anti-cancer agent to a subject, wherein the SRPK1 inhibitor may be administered prior to, simultaneously with and/or after the anti-cancer agent.

Administration of a SRPK1 inhibitor preferably potentiates the effect of the anti-cancer agent. Thus, the effect of treatment with a SRPK1 inhibitor and an anti-cancer agent is additive or synergistic. In one embodiment, the effect of treatment is synergistic.

In one embodiment, administration of the SRPK1 inhibitor allows for administration of the anti-cancer agent at a lower than normal dose, i.e. a dose that would normally be considered a sub-therapeutic dosage.

In one embodiment, administration of the SRPK1 inhibitor enhances the clinical effect of the anti-cancer agent. Clinical effect may be determined by the clinician.

In one embodiment, the anti-cancer agent is a chemotherapeutic agent selected from the group consisting of topoisomerase inhibitors, anti-hormone agents, alkylating agents, antimetabolites, anti-tumour antibiotics, mitotic inhibitors, corticosteroids, targeted anti-cancer agents, differentiating agents, and immunotherapy.

Alkylating Agents

In one embodiment, the chemotherapeutic agent is an alkylating agent. Alkylating agents directly damage DNA (the genetic material in each cell) to keep the cell from reproducing. These drugs work in all phases of the cell cycle and are used to treat many different cancers, including glioblastoma, leukemia, lymphoma, Hodgkin disease, multiple myeloma, and sarcoma, as well as cancers of the lung, breast, and ovary.

Alkylating agents are divided into different classes, including:
Nitrogen mustards: such as mechlorethamine (nitrogen mustard), chlorambucil, cyclophosphamide (Cytoxan®), ifosfamide, and melphalan
Nitrosoureas: such as streptozocin, carmustine (BCNU), and lomustine
Alkyl sulfonates: busulfan
Triazines: dacarbazine (DTIC) and temozolomide (Temodar®)
Ethylenimines: thiotepa and altretamine (hexamethylmelamine)

In one embodiment, the alkylating agent is selected from the group consisting of Nitrogen mustards, Nitrosoureas, Alkyl sulfonates, Triazines, Ethylenimine.

In one embodiment, the alkylating agent is a triazine, such as temozolomide.

The platinum drugs (such as cisplatin, carboplatin, and oxaliplatin) are sometimes grouped with alkylating agents because they kill cells in a similar way. However, in the present context, platinum drugs are not considered alkylating agents.

In one embodiment, the anti-cancer treatment does not comprise or consist of treatment with a metal-based anticancer drug, such as a platinum, ruthenium, gold or titanium-based anticancer drug.

In one embodiment, the anti-cancer treatment does not comprise or consist of treatment with a platinum-based anticancer drug, such as cisplatin, carboplatin, oxaliplatin or nedaplatin.

In one embodiment, an alkylating agent is combined with a SRPK1 inhibitor according to the present disclosure for the treatment of glioblastoma, in particular glioblastoma, which is resistant to treatment with alkylating agents.

In a particular embodiment, the alkylating agent is a triazine, such temozolomide (temodal), the SRPK1 inhibitor is SCO-101 and the cancer to be treated is glioblastoma, in particular temozolomide-resistant glioblastoma.

Antimetabolites

In one embodiment, the chemotherapeutic agent is an antimetabolite. Antimetabolites interfere with DNA and RNA growth by substituting for the normal building blocks of RNA and DNA. These agents damage cells during the S phase, when the cell's chromosomes are being copied. They are commonly used to treat leukemias, cancers of the breast, ovary, and the intestinal tract, e.g. colorectal cancer, pancreatic cancer as well as other types of cancer.

In one embodiment, the antimetabolite is selected from the group consisting of 5-fluorouracil (5-FU), 6-mercaptopurine (6-MP), Capecitabine (Xeloda®), Cytarabine (Ara-C®), Floxuridine, Fludarabine, Gemcitabine (Gemzar®), Hydroxyurea, Methotrexate, Pemetrexed (Alimta®).

In one embodiment, the antimetabolite is 5-fluorouracil (5-FU).

In one embodiment, an anti-metabolite is combined with a SRPK1 inhibitor for the treatment of colorectal cancer, in particular colorectal cancer which is resistant to treatment with anti-metabolites. In one embodiment, the colorectal cancer is metastatic colorectal cancer.

In a particular embodiment, 5-FU is co-administered with a SRPK1 inhibitor, such as SCO-101, for the treatment of colorectal cancer, in particular a 5-FU resistant colorectal cancer. In one embodiment, the colorectal cancer is metastatic colorectal cancer.

Anti-Tumour Antibiotics

These drugs work by altering the DNA inside cancer cells to keep them from growing and multiplying.

In one embodiment, the anti-cancer agent is an anti-tumour antibiotic agent. In other embodiments, the anti-cancer agent is not an anti-tumour antibiotic agent.

In one embodiment, the anti-tumour antibiotic agent is an anthracycline. Anthracyclines are anti-cancer antibiotics that interfere with enzymes involved in DNA replication. These drugs work in all phases of the cell cycle. They are widely used for a variety of cancers. Anthracyclines are also capable of inhibiting topoisomerase II.

Examples of anthracyclines include:
Daunorubicin
Doxorubicin (Adriamycin®)
Epirubicin
Idarubicin

Anti-tumor antibiotics that are not anthracyclines include:
Actinomycin-D
Bleomycin
Mitomycin-C
Mitoxantrone (also acts as a topoisomerase II inhibitor)

Topoisomerase Inhibitors

In one embodiment the chemotherapeutic agent is a topoisomerase inhibitor, which may be a Topoisomerase I inhibitor or a Topoisomerase II inhibitor. These drugs interfere with enzymes called topoisomerases, which help separate the strands of DNA so they can be copied during the S phase. Topoisomerase inhibitors are primarily used to treat colorectal cancer, certain leukemias, as well as lung, ovarian, gastrointestinal, and other cancers.

Topoisomerase inhibitors are grouped according to which type of enzyme they affect.

Topoisomerase I inhibitors include:
Topotecan
Irinotecan (CPT-11). The active metabolite of irinotecan is SN-38.
LMP400
Gimatecan
Topoisomerase II inhibitors include:
Etoposide (VP-16)
Teniposide.
Mitoxantrone (also acts as an anti-tumor antibiotic)
Anthracyclines

In one embodiment the topoisomerase inhibitor is a Topoisomerase I inhibitor, such as Irinotecan or its active metabolite SN-38.

In one embodiment the topoisomerase inhibitor is a Topoisomerase II inhibitor, such as an anthracycline.

In one embodiment a topoisomerase inhibitor, such as a topoisomerase I inhibitor, is combined with a SRPK1 inhibitor for the treatment of colorectal cancer, in particular colorectal cancer which is resistant to treatment with said topoisomerase inhibitor. In one embodiment, the colorectal cancer is metastatic colorectal cancer.

In a particular embodiment, irinotecan/SN-38 is co-administered with a SRPK1 inhibitor, such as SCO-101, for the treatment of colorectal cancer, in particular an irinotecan/SN-38 resistant colorectal cancer. In one embodiment, the colorectal cancer is metastatic colorectal cancer.

Mitotic Inhibitors

In one embodiment, the chemotherapeutic agent is a mitotic inhibitor. Mitotic inhibitors are often plant alkaloids and other compounds derived from natural products. They work by stopping mitosis in the M phase of the cell cycle but can damage cells in all phases by keeping enzymes from making proteins needed for cell reproduction.

Examples of mitotic inhibitors include:
Taxanes: paclitaxel (Taxol®), docetaxel (Taxotere®) and abraxane
Epothilones: ixabepilone (Ixempra®)
Vinca alkaloids: vinblastine (Velban®), vincristine (Oncovin®), and vinorelbine (Navelbine®)
Estramustine (Emcyt®)

Mitotic inhibitors are used to treat many different types of cancer including breast, pancreatic, lung, myelomas, lymphomas, and leukemias.

In one embodiment, the mitotic inhibitor is a taxane, such as paclitaxel, docetaxel or abraxane.

In one embodiment, a mitotic inhibitor is combined with a SRPK1 inhibitor for the treatment of breast cancer, in particular breast cancer which is resistant to treatment with mitotic inhibitors. In one embodiment, the breast cancer is metastatic breast cancer.

In a particular embodiment, a taxane, such as paclitaxel or docetaxel or Abraxane is co-administered with a SRPK1 inhibitor, such as SCO-101, for the treatment of breast cancer, in particular a paclitaxel or docetaxel or Abraxane resistant breast cancer. In one embodiment the breast cancer is metastatic breast cancer.

Corticosteroids

In one embodiment the chemotherapeutic agent is a corticosteroid. Corticosteroids, often simply called steroids, are natural hormones and hormone-like drugs that are useful in the treatment of many types of cancer, as well as other illnesses.

When these drugs are used as part of cancer treatment, they are considered chemotherapeutic agents.

Examples of corticosteroids include:
Prednisone
Methylprednisolone (Solumedrol®)
Dexamethasone (Decadron®).

Steroids are also commonly used to help prevent nausea and vomiting caused by chemotherapy. They are used before chemotherapy to help prevent severe allergic reactions, too.

In some embodiments, the chemotherapeutic agent is not a corticosteroid.

Other Chemotherapeutic Agents

Some chemotherapeutic agents act in slightly different ways and do not fit well into any of the other categories. Examples include drugs like L-asparaginase, which is an enzyme, and the proteasome inhibitor bortezomib (Velcade®).

In one embodiment, the chemotherapeutic agent is not a metal-based anticancer drug, such as a platinum, ruthenium, gold or titanium-based anticancer drug. In one embodiment, the chemotherapeutic agent is not a platinum-based anticancer drug, such as cisplatin, carboplatin, oxaliplatin or nedaplatin.

Targeted Anti-Cancer Agents

In one embodiment, the chemotherapeutic agent is a targeted anti-cancer agent, such as an antibody-based agent, which acts on a well-defined target or biologic pathway.

Examples of targeted agents include:
Imatinib (Gleevec®)
Gefitinib (Iressa®)
Sunitinib (Sutent®)
Bortezomib (Velcade®)
Anti-EGF receptor (Cetuximab)

In one embodiment the targeted anti-cancer agent is an anti-angiogenesis agent, such as an anti-VEGF agent. For instance, the anti-angiogenesis agent may be a humanised anti-VEGF monoclonal antibody, such as Avastin (Bevacizumab). If the anti-cancer agent of the present disclosure is an anti-angiogenesis agent, the anti-angiogenesis agent is not a SRPK1 inhibitor such as SCO-101.

In some embodiments, the chemotherapeutic agent is not a targeted anti-cancer agent.

Differentiating Agents

These drugs act on the cancer cells to make them mature into normal cells. Examples include the retinoids, tretinoin (ATRA or Atralin®) and bexarotene (Targretin®), as well as arsenic trioxide (Arsenox®).

In one embodiment, the chemotherapeutic agent is a differentiating agent. In some embodiments, the chemotherapeutic agent is not a differentiating agent.

Anti-Hormone Agents

In one embodiment, the chemotherapeutic agent is an agent for anti-hormone therapy. Drugs in this category are sex hormones, or hormone-like drugs, that change the action or production of female or male hormones, e.g. by reducing endogenous production of hormones or by blocking steroid hormone receptors. They are used to slow the growth of breast, prostate, and endometrial (uterine) cancers, which normally grow in response to natural sex hormones in the body. These cancer endocrine treatments do not work in the same ways as standard chemotherapy drugs. They work by making the cancer cells unable to use the hormone they need to grow, or by preventing the body from making the hormone.

Examples of anti-hormone therapy include:
Anti-estrogens: fulvestrant (Faslodex®), tamoxifen, and toremifene (Fareston®), clomifene, and raloxifene.
Anti-progestogens: mifepristone, ulipristal acetate, aglepristone, lilopristone and onapristone
Anti-androgens: bicalutamide (Casodex®), flutamide (Eulexin®), and nilutamide (Nilandron®)
Aromatase inhibitors: anastrozole (Arimidex®), exemestane (Aromasin®), and letrozole (Femara®)
Progestins: megestrol acetate (Megace®)
Estrogens
Gonadotropin-releasing hormone (GnRH), also known as luteinizing hormonereleasing hormone (LHRH) agonists or analogs: leuprolide (Lupron®) and goserelin (Zoladex®).

In one embodiment, the anti-cancer treatment comprises anti-estrogen treatment. Anti-estrogens, also known as estrogen receptor antagonists or estrogen receptor blockers, are a class of drugs, which prevent estrogens like estradiol from mediating their biological effects in the body.

In one embodiment, the chemotherapeutic agent is an anti-hormonal agent, such as an anti-estrogen for example tamoxifen, an aromatase inhibitor, a selective estrogen receptor modulator (SERM) such as Fulvestrant, or an anti-progestogen.

In one embodiment, the anti-estrogen is fulvestrant.

In one embodiment, the anti-estrogen is tamoxifen.

In one embodiment, the anti-cancer agent comprises anti-progestine gen treatment. Anti-progestines, or anti-progestins, also known as progesterone receptor antagonists or progesterone blockers, are a class of drugs which prevent progestogens like progesterone from mediating their biological effects in the body.

Examples of anti-progestogens include mifepristone, ulipristal acetate, aglepristone, lilopristone and onapristone.

In one embodiment, the anti-cancer agent comprises an anti-androgen agent. Said agents, anti-androgens, also known as androgen receptor antagonists or testosterone blockers, are a class of drugs, which prevent androgens like testosterone and dihydrotestosterone (DHT) from mediating their biological effects in the body.

In some embodiments, the chemotherapeutic agent is not an anti-hormone agent.

Anti-hormone therapy is particularly useful for treatment of steroid hormone receptor positive cancers, for example anti-estrogens are used for treatment of ER positive breast or uterine cancer.

In one embodiment, an anti-estrogen is co-administered with a SRPK1 inhibitor, such as SCO-101, for the treatment of an ER positive cancer, such as an ER positive breast cancer. In one embodiment, the breast cancer is metastatic breast cancer. In one embodiment, an anti-estrogen is co-administered with a SRPK1 inhibitor, such as SCO-101, for the treatment of an anti-estrogen resistant cancer.

In one embodiment, an anti-progestogen is co-administered with a SRPK1 inhibitor, such as SCO-101, for the treatment of a PR positive cancer, such as a PR positive breast cancer. In one embodiment, the breast cancer is metastatic breast cancer.

In one embodiment, an anti-androgen is co-administered with a SRPK1 inhibitor, such as SCO-101, for the treatment of an AR positive cancer, such as an AR positive prostate cancer. In one embodiment, the prostate cancer is metastatic prostate cancer.

Immunotherapy

In one embodiment, the chemotherapeutic agent is an immunotherapy agent. Immunotherapy drugs are given to people with cancer to help their immune systems recognize and attack cancer cells.

There are different types of immunotherapy. Active immunotherapies stimulate the body's own immune system to fight the disease. Passive immunotherapies do not rely on the body to attack the disease; they're immune system components (such as antibodies) created outside the body and given to fight the cancer.

Examples of active immunotherapies include:
Monoclonal antibody therapy, such as rituximab (Rituxan®) and alemtuzumab (Campath®)
Non-specific immunotherapies and adjuvants (other substances or cells that boost the immune response), such as BCG, interleukin-2 (IL-2), and interferon-alfa
Immunomodulating drugs, such as thalidomide and lenalidomide (Revlimid®)

In one embodiment, the chemotherapeutic agent is a PD-1 or PD-L1 inhibitor, such as an antibody capable of inhibiting PD-1 or PD-L1.

Cancer vaccines are a type of active specific immunotherapy.

In some embodiments, the chemotherapeutic agent is not an immunotherapy agent.

Radiation Therapy

In one embodiment, the combination treatment further comprises radiation therapy. Radiation therapy is therapy using ionizing radiation, generally as part of cancer treatment to control or kill malignant cells. Radiation therapy may be curative in a number of types of cancer if they are localized to one area of the body. It may also be used as part of adjuvant therapy, to prevent tumour recurrence after surgery to remove a primary malignant tumour (for example, early stages of breast cancer). Radiation therapy is synergistic with chemotherapy, and can be used before, during, and after chemotherapy in susceptible cancers. Doses and treatment schedules of radiation therapy vary depending on the type and stage of cancer being treated and can be determined by the clinician.

Combination of Kinase Inhibitors

In some embodiments, a further kinase inhibitor, such as Regorafinib, is co-administered with the SRPK1 inhibitor according to the present disclosure for the treatment of cancer in a patient with elevated expression and/or activity of SRPK1.

Inhibition of Drug Efflux Pumps

In one embodiment, the SRPK1 inhibitor as disclosed herein also inhibits a drug efflux pump. Overexpression of drug efflux pumps, also known as ABC transporter efflux pumps are among the main reasons for the development of multi-drug resistant tumours and bacterial and fungal infections. Hence, inhibition of the drug efflux pumps may benefit the outcome of treatment with a further medicament, wherein the further medicament is a substrate for the drug efflux pump. The substrate for the efflux pump according to the present disclosure may be a chemotherapeutic agent.

In one embodiment, the chemotherapeutic agent which is a substrate for a drug efflux pump is a topoisomerase I inhibitor, such as a topoisomerase I inhibitor selected from the group topotecan, irinotecan (CPT-11) and SN-38.

In one embodiment, the chemotherapeutic agent which is a substrate for a drug efflux pump is a topoisomerase II inhibitor, such as an anthracycline.

In one embodiment, the chemotherapeutic agent which is a substrate for a drug efflux pump is a taxane, such as docetaxel, paclitaxel or abraxane.

The drug efflux pumps may be but are not limited to P-glycoprotein (P-gp/ABCB1), multidrug resistance-associated protein 2 (MRP2/ABCC2), and breast cancer resistance protein (BCRP/ABCG2).

Determining Expression and/or Activity of SRPK1

Expression levels of SRPK1 may be determined by any methods suitable for determining expression levels at the mRNA and/or protein level. Suitable methods for protein expression measurements include but are not limited to: Immunohistochemistry, Western Blotting, immunocytology and ELISA. For mRNA measurements, suitable methods include but are not limited to: RT-PCR, QPCR and in situ hybridization.

Activity of SRPK1 may be determined by any method known to a person of skill, e.g. by a radioactive filter binding assay using 33P ATP as described previously (Hastie, et al 2006. Nat Protoc. 2006; 1(2):968-71; Bain, et al 2007. Biochem J. 2007 Dec. 15; 408(3):297-315; the teachings of which are incorporated herein by reference). This method is sensitive, accurate and provides a direct measure of kinase enzyme activity.

All human cells express SRPK1. When defining an increased or elevated level of SRPK1 in a cell or a tissue, one can compare SRPK1 expression and/or activity in non-diseased tissue/cells with expression and/or activity in the diseased tissue/cells in question. For example, when defining SRPK-1 expression levels, being at the protein or mRNA level, the comparison could be between normal breast tissue expression and expression in breast cancer cells in the individual patient. Alternatively, by measuring SRPK1 levels in a large number of healthy tissues/cells, a control value for normal tissue/cells can be established. Any increased level compared to the value in the normal cells/tissue will be considered as elevated.

In one embodiment, the elevated expression and/or activity of SRPK1 is in diseased tissue or diseased cells. In one embodiment, the sample is a biopsy sample or a tissue resectate. In one embodiment, the sample is a body fluid sample comprising diseased cells, e.g. wherein the sample is a blood sample or a spinal fluid sample. In a further embodiment, the control sample is obtained from the same subject as the sample comprising diseased tissue or diseased cells and is a sample comprising healthy tissue or healthy cells of the same origin as the diseased tissue or diseased cells. In one embodiment the control sample is obtained from one or more healthy subjects and comprises healthy tissue of the same origin as the diseased tissue. In one embodiment, the diseased tissue is cancerous tissue. In one embodiment, the diseased cells are cancer cells such as circulating tumor cells. In one embodiment, the expression level of SRPK1 is measured at the mRNA and/or the protein level.

Ranges for Elevated Expression and/or Activity

In an aspect of the present disclosure, the elevated expression and/or activity of SRPK1 in diseased tissue or diseased cells is above 1.2, quantified relative to the expression level and/or activity of SRPK1 in a control sample comprising non-diseased tissue or non-diseased cells, wherein the expression level of SRPK1 in the control sample is set to 1.

In a further aspect of the present disclosure, the elevated expression and/or activity of SRPK1 in diseased tissue or diseased cells is at least 1.8, such as at least 2, such as at least 2.5, such as at least 3, such as at least 3.5, such as at least 4.5, such as at least 5.5, such as at least 6.5, such as at least 7.5, such as at least 8.5, such as at least 9.5, such as at least 10 relative to the expression level and/or activity of SRPK1 in the control sample. In one embodiment the elevated expression and/or activity of SRPK1 in diseased tissue or diseased cells is at least 3 times that of the expression and/or activity in the control sample.

Companion Diagnostics

The present invention further relates to identifying subjects which may benefit from treatment with the SPRK1 inhibitors defined herein.

In an aspect of the present disclosure, the expression level and/or activity of SRPK1 is measured in a subject as described herein, and if the expression level and/or activity of SPRK1 is elevated, a SRPK1 inhibitor according to the present disclosure is administered to said subject.

In one embodiment of the present disclosure, a method for treatment of a disease characterized by an elevated expression and/or activity of Serine Arginine Protein Kinase 1 (SRPK1) is provided, comprising administering to a subject an effective amount of the SRPK1 inhibitor as defined herein and optionally a further medicament, wherein a sample comprising diseased tissue or diseased cells obtained from said subject comprises an elevated expression and/or activity of SPRK1 relative to the expression level and/or activity of SPRK1 in a control sample.

SRPK1 Inhibitors

According to the present disclosure, the selective SRPK1 inhibitor is a SRPK1 inhibitor of formula I,

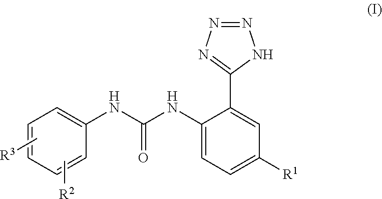

or a pharmaceutically acceptable salt thereof,

wherein

R¹, R² and R³ independently of each other represent hydrogen, halo, trifluoromethyl, nitro, alkyl, alkylcarbonyl, —NR^{a}R^{b}, —NR^{a}—CO—R^{b}, phenyl or heteroaryl; which phenyl is optionally substituted with halo, trifluoromethyl, nitro, —CO—NHR^{c}, —CO—O—R^{c} or —CO—NR′R″;

wherein R^{c} is hydrogen, alkyl, or phenyl;

R′ and R″ independently of each other are hydrogen or alkyl; or

R′ and R″ together with the nitrogen to which they are attached form a 5- to 7-membered heterocyclic ring, which ring may optionally comprise as a ring member, one oxygen atom, and/or one additional nitrogen atom, and/or one carbon-carbon double bond, and/or one carbon-nitrogen double bond;

and which heterocyclic ring may optionally be substituted with alkyl;

R^{a} and R^{b} independently of each other are hydrogen or alkyl.

In some embodiments, R¹ of formula (I) represents halo. In one embodiment, R² and R³ independently of each other represent halo or trifluoromethyl.

In one embodiment, the SRPK1 inhibitor of formula (I) is selected from:
N-4-Nitrophenyl-N′-[4-bromo-2-(1-H-tetrazol-5-yl)phenyl] urea;
N-3,5-Di(trifluoromethyl)phenyl-N′-[4-bromo-2-(1-H-tetrazol-5-yl)phenyl] urea;
N-3-Trifluoromethylphenyl-N′-[4-(3-nitrophenyl)-2-(1-H-tetrazol-5-yl)phenyl] urea;
N-3-Trifluoromethylphenyl-N′-[4-(4-anilinocarbonylphenyl)-2-(1-H-tetrazol-5-yl)phenyl]urea;
N-3-Trifluoromethylphenyl-N′-[4-(4-trifluoromethylphenyl)-2-(1-H-tetrazol-5-yl)phenyl]urea;
N-(3-Trifluoromethyl-phenyl)-N′-[2-(1-H-tetrazol-5-yl)-phenyl] urea;
N-(3-Trifluoromethyl-phenyl)-N′-[4-bromo-2-(1-H-tetrazol-5-yl)-phenyl] urea;
N-(3-Trilfuoromethyl-phenyl)-N′-[4-phenyl-2-(1-H-tetrazol-5-yl)-phenyl] urea;
N-(3-Chloro-phenyl)-N′-[2-(1-H-tetrazol-5-yl)-phenyl] urea;
N-(3-Trifluoromethyl-phenyl)-N′-[4-amino-2-(1-H-tetrazol-5-yl)-phenyl] urea;
N-(3-Trifluoromethyl-phenyl)-N′-[4-acetylamino-2-(1-H-tetrazol-5-yl)-phenyl] urea;
N-(3-Trifuoromethyl-phenyl)-N′-[4-carbamoyl-2-(1-H-tetrazol-5-yl)-phenyl] urea;
N-(3-Trifluoromethyl-phenyl)-N′-[4-(N″,N″-dimethylcarbamoyl)-2-(1-H-tetrazol-5-yl)-phenyl] urea;
3′-(1-H-tetrazol-5-yl)-4′-[3-(3-trifluoromethyl-phenyl)-ureido]-biphenyl-4-carboxylic acid;
N-(Biphenyl-4-yl)-N′-[2-(1-H-tetrazol-5-yl)-phenyl] urea;
N-(Biphenyl-3-yl)-N′-[2-(1-H-tetrazol-5-yl)-phenyl] urea;
N-(3-Acetyl-phenyl)-N′-[2-(1-H-tetrazol-5-yl)-phenyl] urea;
N-(Biphenyl-3-yl)-N′-[2-(1-H-tetrazol-5-yl)-phenyl] urea;
N-[3-(Pyridin-3-yl)-phenyl]-N′-[2-(1-H-tetrazol-5-yl)-phenyl] urea;
N-(3-Bromo-phenyl)-N′-[4′-(4-methyl-piperazine-1-carbonyl)-3-(1-H-tetrazol-5-yl)-biphenyl-4-yl] urea;
N-(3,5-Dichlorophenyl)-N′-[4-bromo-2-(1-H-tetrazol-5-yl)-phenyl] urea;
N-(3,4-Dichlorophenyl)-N′-[4-bromo-2-(1-H-tetrazol-5-yl)-phenyl] urea;
N-(2-Trifluoromethyl-phenyl)-N′-[4-bromo-2-(1-H-tetrazol-5-yl)-phenyl] urea;
N-(2-Fluorophenyl)-N′-[4-bromo-2-(1-H-tetrazol-5-yl)-phenyl] urea;
N-(2-Ethylphenyl)-N′-[4-bromo-2-(1-H-tetrazol-5-yl)-phenyl] urea;
   or a pharmaceutically acceptable salt thereof.

In one embodiment, the SRPK1 inhibitor of formula (I) is selected from:
N-4-Nitrophenyl-N′-[4-bromo-2-(1-H-tetrazol-5-yl)phenyl] urea;
N-3,5-Di(trifluoromethyl)phenyl-N′-[4-bromo-2-(1-H-tetrazol-5-yl)phenyl] urea;
N-3-Trifluoromethylphenyl-N′-[4-(3-nitrophenyl)-2-(1-H-tetrazol-5-yl)phenyl] urea;
N-3-Trifluoromethylphenyl-N′-[4-(4-anilinocarbonylphenyl)-2-(1-H-tetrazol-5-yl)phenyl]urea;
N-3-Trifluoromethylphenyl-N′-[4-(4-trifluoromethylphenyl)-2-(1-H-tetrazol-5-yl)phenyl]urea;
   or a pharmaceutically acceptable salt thereof.

In a particular embodiment, the SRPK1 inhibitor is an SRPK1 inhibitor of formula (III):

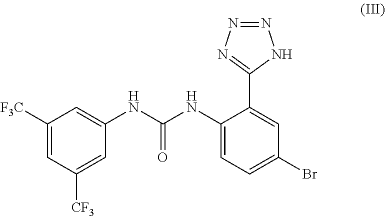 [SMILES: O=C(Nc1cc(C(F)(F)F)cc(C(F)(F)F)c1)Nc1ccc(Br)cc1-c1nnn[nH]1]

or a pharmaceutically acceptable salt thereof.

The SRPK1 inhibitor of formula (III) is SCO-101.

In one embodiment, the SRPK1 inhibitor has an IC50 against SRPK1 of 10 μM or less, such as 8 μM or less, for example 6 μM or less; such as 5 μM or less, for example 4 μM or less, such as 3 μM or less, for example 1 μM or less, such as 0.5 μM or less, for example 0.1 μM or less, such as 10 nM or less, for example 5 nM or less, preferably wherein the IC50 is 5 μM or less.

The SRPK1 inhibitor of the present disclosure is preferably a selective inhibitor of SRPK1. Thus, in one embodiment, the SRPK1 inhibitor is capable of inhibiting the mean activity of SRPK1 to less than 10% of the activity of a control sample, e.g. a DMSO control, preferably to less than 8%, such as less than 7%, such as less than 6%, such as less than 5% of control. The selective SRPK1 inhibitor of the present disclosure may have some capability to inhibit other kinases, but preferably does not inhibit the activity of any other kinases to more than 20% of control, more preferably no more than 25% of control, even more preferably to more than 30% of control.

### Definition of Substituents

In the context of the present disclosure halo represents fluoro, chloro, bromo or iodo.

In the context of present disclosure, an alkyl group designates a univalent saturated, straight or branched hydrocarbon chain. The hydrocarbon chain preferably contain of from one to six carbon atoms (C₁₋₆-alkyl), including pentyl, isopentyl, neopentyl, tertiary pentyl, hexyl and isohexyl. In one embodiment alkyl represents a C₁₋₄-alkyl group, including butyl, isobutyl, secondary butyl, and tertiary butyl. In another embodiment of this invention, alkyl represents a C₁₋₃-alkyl group, which may in particular be methyl, ethyl, propyl or isopropyl.

In the context of present disclosure, a heteroaryl group designates an aromatic mono-, bi- or poly-heterocyclic group, which holds one or more heteroatoms in its ring structure. Preferred heteroatoms include nitrogen (N), oxygen (O), and sulphur (S). Preferred monocyclic heteroaryl groups of the invention include aromatic 5- and 6 membered heterocyclic monocyclic groups, including furanyl, in particular 2- or 3-furanyl; thienyl, in particular 2 or 3-thienyl; pyrrolyl (azolyl), in particular 1,2 or 3-pyrrolyl; oxazolyl, in particular oxazol-2,4 or 5-yl; thiazolyl, in particular thiazol-2,4 or 5-yl; imidazolyl, in particular 1,2 or 4-imidazolyl; pyrazolyl, in particular 1,3 or 4-pyrazolyl; isoxazolyl, in particular isoxazol-3,4 or 5-yl; isothiazolyl, in particular isothiazol-3,4 or 5-yl; oxadiazolyl, in particular 1,2,3-, 1,2,4-, 1,2,5- or 1,3,4-oxadiazol-3,4 or 5-yl; triazolyl, in particular 1,2,3-, 1,2,4-, 2,1,3- or 4,1,2-triazolyl; thiadiazolyl, in particular thiadiazol-3,4 or 5-yl; pyridinyl, in particular 2,3 or 4-pyridinyl; pyridazinyl, in particular 3 or 4-pyridazinyl; pyrimidinyl, in particular 2,4 or 5-pyrimidinyl; pyrazinyl, in particular 2 or 3-pyrazinyl; and triazinyl, in particular 1,2, 3-, 1,2,4- or 1,3,5-triazinyl. 5- to 7-membered heterocyclic rings comprising one nitrogen atom include for example, but not limited to, pyrolidine, piperidine, homopiperidine, pyrroline, tetrahydropyridine, pyrazolidine, imidazolidine, piperazine, homopiperazine, and morpholine.

Administration

In one embodiment, the SRPK1 inhibitor as disclosed herein is in the form of tablets or capsules for oral administration. In one embodiment, the SRPK1 inhibitor is in the form of a liquid for intravenous administration or continuous infusion. In one embodiment, the composition is administered topically.

Subjects

The subject according to the present disclosure may be a mammal, preferably a human being. Treatment of animals, such as mice, rats, dogs, cats, horses, cows, sheep and pigs, is, however, also within the scope of the present context. The subject to be treated can be of any age, i.e. an infant, a child, an adolescent or an adult.

### EXAMPLES

### Example 1: SCO-101 is a Selective SRPK1 Inhibitor

### BACKGROUND

Many novel anti-cancer drugs are acting by inhibition of specific protein kinases. Therefore, we investigated a panel of protein kinase in vitro to test for any inhibition mediated by SCO-101.

Materials and Methods

The kinase activity screening was done at MRC in Dundee. The screen was a so-called “Premier Screen” with 140 kinases. More specific information can be found here: http://www.kinase-screen.mrc.ac.uk/services/premier-screen

The method is based on a radioactive filter binding assay using 33P ATP (Hastie, et al 2006. Nat Protoc. 2006; 1(2):968-71; Bain, et al 2007. Biochem J. 2007 Dec. 15; 408(3):297-315). This method is sensitive, accurate and provides a direct measure of kinase enzyme activity. A concentration of 10 μM SCO-101 was used.

Results

In the table below, the data is portrayed as the mean percentage activity of assay duplicates and a standard deviation. These percentage activities were calculated in comparison to DMSO controls. 10 μM SCO-101 reduced the activity of SRPK1 to 6% of the DMSO control.

### CONCLUSIONS

Of the tested protein kinases, SRPK1 was the most influenced by SCO-101, and the activity was reduced to 6% of activity seen in the DMSO control (Table 1). Other kinases, such as TRkA were also inhibited somewhat (25% of control), however not as strongly as SRPK1. SCO-101 was also found to increase the activity of several kinases, e.g. CHK1 (195% of DMSO control). Based on the kinase activity screen, we conclude that SCO-101 is a selective SRPK1 inhibitor.

TABLE 1

| | Kinase activity | |
|---|---|---|
| | (percentage of DMSO | Standard |
| | control) | deviation |
| SRPK1 | 6 | 6 |
| TrkA | 25 | 12 |
| PAK4 | 38 | 4 |
| SIK3 | 39 | 0 |
| NUAK1 | 49 | 7 |
| PIM3 | 49 | 2 |
| TESK1 | 54 | 5 |
| CAMK1 | 57 | 14 |
| DYRK3 | 59 | 3 |
| PLK1 | 59 | 3 |
| EPH-A2 | 60 | 5 |
| Aurora B | 61 | 12 |
| PIM1 | 61 | 12 |
| SYK | 62 | 2 |
| PDGFRA | 62 | 11 |
| p38a MAPK | 63 | 17 |
| RSK2 | 64 | 4 |
| VEG-FR | 66 | 18 |
| DDR2 | 68 | 5 |
| PRAK | 69 | 6 |
| PIM2 | 72 | 2 |
| MARK1 | 74 | 6 |
| IKKe | 75 | 1 |
| SmMLCK | 77 | 1 |
| DYRK2 | 78 | 1 |
| BTK | 78 | 4 |
| SIK2 | 81 | 9 |
| BRSK2 | 81 | 3 |
| TSSK1 | 83 | 6 |
| PAK2 | 83 | 6 |
| PKBb | 84 | 4 |
| MAPKAP-K3 | 85 | 9 |
| ABL | 85 | 1 |
| CLK2 | 86 | 0 |
| EPH-B4 | 86 | 0 |
| PHK | 88 | 18 |
| TTK | 88 | 4 |
| NEK6 | 90 | 3 |
| ERK5 | 90 | 0 |
| MARK4 | 90 | 0 |
| MNK1 | 91 | 11 |
| GCK | 92 | 8 |
| S6K1 | 92 | 15 |
| ERK8 | 93 | 0 |
| OSR1 | 94 | 8 |
| CAMKKb | 94 | 21 |
| MKK2 | 94 | 1 |
| BRK | 94 | 15 |
| Aurora A | 94 | 9 |
| TTBK1 | 94 | 15 |
| TIE2 | 95 | 18 |
| ULK2 | 95 | 15 |
| EF2K | 95 | 8 |
| MELK | 95 | 13 |
| LKB1 | 96 | 0 |
| AMPK (hum) | 97 | 2 |
| PKCγ | 97 | 5 |
| MLK1 | 98 | 7 |
| HER4 | 98 | 10 |
| HIPK1 | 98 | 15 |
| IR | 98 | 2 |
| YES1 | 99 | 12 |
| GSK3b | 99 | 2 |
| ZAP70 | 99 | 10 |
| MARK3 | 99 | 8 |
| IKKb | 99 | 16 |
| MAPKAP-K2 | 100 | 2 |
| STK33 | 100 | 6 |
| RSK1 | 100 | 2 |
| MARK2 | 100 | 9 |
| MSK1 | 101 | 3 |
| ROCK 2 | 101 | 7 |
| PKCz | 101 | 5 |
| EPH-A4 | 101 | 18 |
| EIF2AK3 | 103 | 7 |
| SGK1 | 103 | 2 |
| Lck | 103 | 2 |
| MNK2 | 104 | 9 |
| CDK9-Cyclin T1 | 104 | 17 |
| PINK | 104 | 8 |
| MEKK1 | 105 | 14 |
| JNK1 | 105 | 0 |
| WNK1 | 105 | 11 |
| JNK2 | 105 | 2 |
| Src | 105 | 24 |
| PKA | 106 | 2 |
| CSK | 106 | 26 |
| TAK1 | 107 | 3 |
| CK1δ | 107 | 4 |
| CK2 | 107 | 24 |
| MAP4K5 | 108 | 16 |
| MKK6 | 108 | 3 |
| PDK1 | 108 | 4 |
| TTBK2 | 109 | 9 |
| CK1γ2 | 109 | 10 |
| MLK3 | 110 | 25 |
| p38g MAPK | 110 | 4 |
| TGFBR1 | 111 | 7 |
| JNK3 | 112 | 16 |
| DYRK1A | 112 | 5 |
| IRAK4 | 112 | 22 |
| DAPK1 | 112 | 4 |
| CDK2-Cyclin A | 113 | 5 |
| MAP4K3 | 113 | 10 |
| p38d MAPK | 113 | 3 |
| CHK2 | 114 | 2 |
| ULK1 | 114 | 5 |
| EPH-B3 | 114 | 4 |
| p38b MAPK | 115 | 5 |
| IRAK1 | 115 | 5 |
| MPSK1 | 115 | 8 |
| PKCa | 116 | 4 |
| TAO1 | 116 | 12 |
| EPH-B2 | 117 | 1 |
| MST4 | 117 | 23 |
| MKK1 | 118 | 1 |
| ERK2 | 119 | 5 |
| PRK2 | 120 | 19 |
| RIPK2 | 121 | 15 |
| JAK3 | 121 | 13 |
| HIPK3 | 121 | 13 |
| PAK5 | 121 | 15 |
| TLK1 | 122 | 10 |
| PKD1 | 122 | 13 |
| PAK6 | 123 | 5 |
| FGF-R1 | 123 | 13 |
| EPH-B1 | 124 | 19 |
| BRSK1 | 124 | 11 |
| PKBa | 126 | 5 |
| TBK1 | 126 | 28 |
| IGF-1R | 128 | 2 |
| IRR | 131 | 4 |
| NEK2a | 134 | 9 |
| MST3 | 135 | 6 |
| ASK1 | 136 | 4 |
| ERK1 | 137 | 1 |
| HIPK2 | 139 | 16 |
| MST2 | 146 | 14 |
| MINK1 | 173 | 4 |
| CHK1 | 195 | 29 |

### Example 2: SCO-101 Determination of IC50

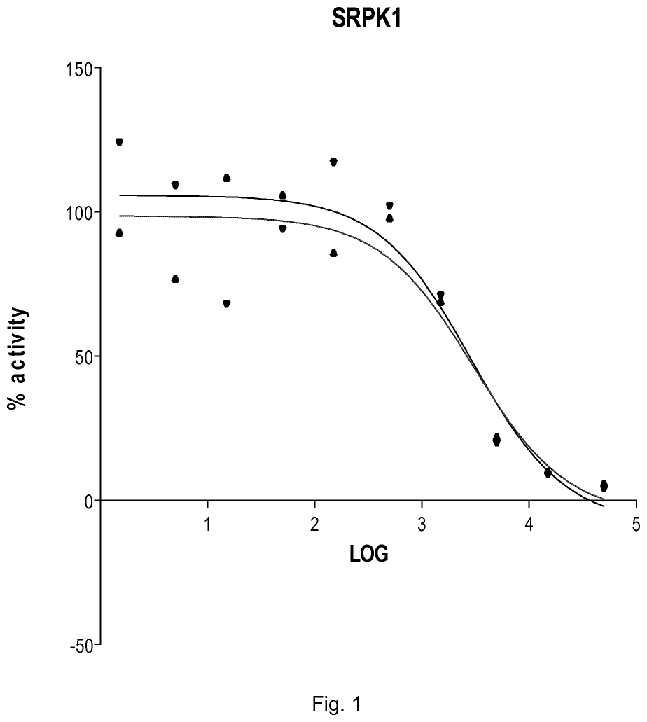

This experiment was technically carried out as described in example 1, also done by MRC in Dundee. However, in this experiment 10 different concentrations of SCO-101 was applied in order to determine the half maximal inhibitory concentration (IC50). The following concentrations (in nM) were applied: 1.5, 5, 15, 50, 150, 500, 1500, 5000, 5000 and 50000. This experiment generated a dose response curve (FIG. 1), which confirmed the SCO-101 mediated inhibition of SRPK1. The IC50 value was determined to be 3.0 μM (Table 2).

TABLE 2

| | IC50(μM) | IC50(μM) | mean IC50 (uM) | s.d. |
|---|---|---|---|---|
| SRPK1 | 2.9 | 3.0 | 3.0 | 0.1 |

### Example 3: Validation of SN38 Resistant Model System in Colon Cancer Cells

SN38 was applied to a SN38 sensitive colon cancer cell line (“HT29 parental”) and to a SN38 resistant colon cancer cell line (“HT29 SN38 resistant”).

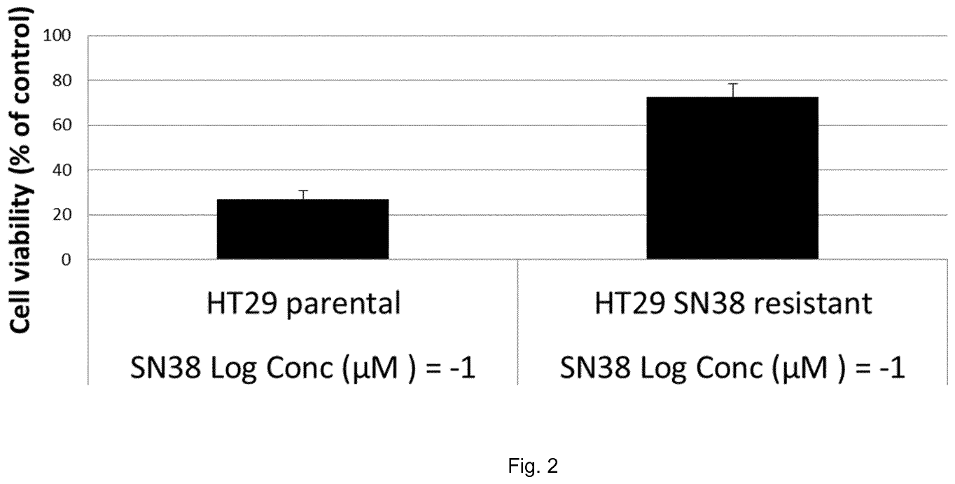

The Log Concentration (μM) used are—1 for SN38. Data represents cell viability data (MTT assay) and cells were incubated with drugs for 72 hours. Data shows that the SN38 caused a reduction of cell viability to 27% of the untreated control cells, whereas the viability in the SN38 resistant HT29 cells was only reduced to 73% of untreated control cells (FIG. 2).

### Example 4: SCO-101 Causes Re-Sensitization to SN38 in a SN38 Resistant Colorectal Cancer Cell Line

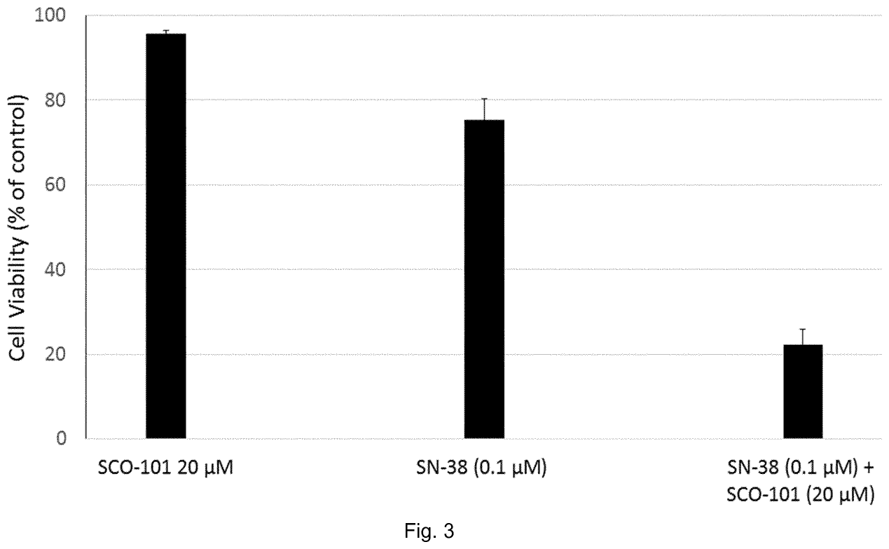

SN38 and SCO-101 were applied to a SN38 resistant colon cancer cell line (“HT29 SN38 resistant” either alone or in combination. The concentrations used are 0.1 μM for SN38 and 20 μM for SCO-101. Data represents cell viability data (MTT assay) and cells were incubated with drugs for 72 hours. Data shows that the SN38 caused a reduction of cell viability to 75% of the untreated control cells, SCO-101 caused a reduction of cell viability to 96% of the untreated control cells and the combination of SN38 and SCO-101 caused a reduction of cell viability to 22% of the untreated control cells. These data demonstrates the ability of SCO-101 to cause re-sensitization of SN38 resistant HT29 cells to SN38 (FIG. 3).

### Example 5: Two Different Synthetic SRPK1 Inhibitors Cause Re-Sensitization to SN38 in a SN38 Resistant Colorectal Cancer Cell Line

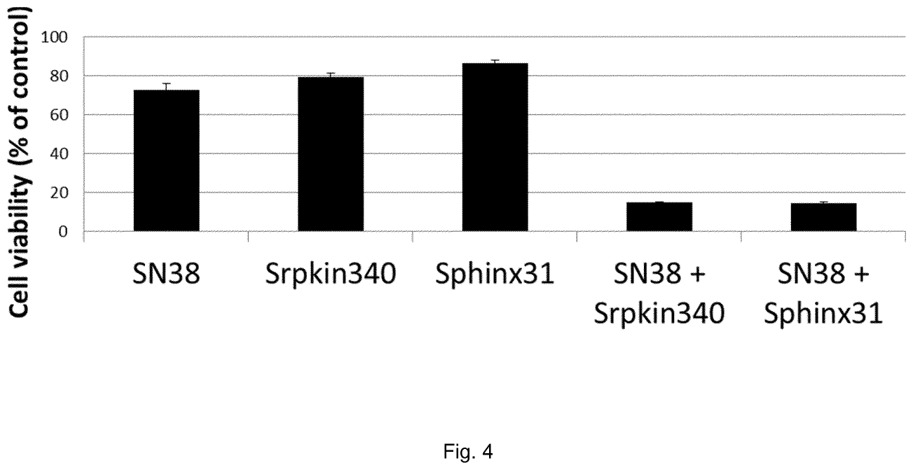

The Log Concentrations (μM) used are—1 for SN38, and 1.5 for both Srpkin340 and Sphinx 31. Data represents cell viability data (MTT assay) and cells were incubated with drugs for 72 hours. Data shows that two different SRPK1 inhibitors (Srpkin340 and Sphinx31) both can restore the SN38 sensitivity in SN38 resistant HT29 colon cancer cells (FIG. 4). Based on these results, we conclude that the ability of SCO-101 to re-sensitize chemotherapy-resistant cancer cells is likely mediated through its ability to selectively inhibit the activity of SRPK1.

1. A method of treating a disease comprising administering to a subject an effective amount of a composition comprising a SRPK1 inhibitor of formula I, 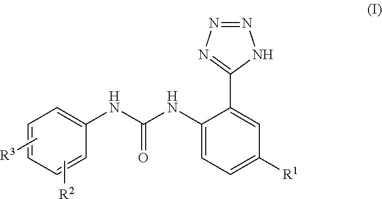
or a pharmaceutically acceptable salt thereof,
wherein
R¹, R² and R³ independently of each other represent hydrogen, halo, trifluoromethyl, nitro, alkyl, alkylcarbonyl, —NR^{a}R^{b}, —NR^{a}—CO—R^{b}, phenyl or heteroaryl;
which phenyl is optionally substituted with halo, trifluoromethyl, nitro, —CO—NHR^{c}, —CO—O—R^{c} or —CO—NR′R″;
wherein R^{c} is hydrogen, alkyl, or phenyl;
R′ and R″ independently of each other are hydrogen or alkyl; or
R′ and R″ together with the nitrogen to which they are attached form a 5- to 7-membered heterocyclic ring, which ring may optionally comprise as a ring member, one oxygen atom, and/or one additional nitrogen atom, and/or one carbon-carbon double bond, and/or one carbon-nitrogen double bond;
and which heterocyclic ring may optionally be substituted with alkyl;
R^{a} and R^{b} independently of each other are hydrogen or alkyl;
wherein the subject is characterized by an elevated expression and/or activity of Serine Arginine Protein Kinase 1 (SRPK1).

2. The method according to claim 1, wherein the elevated expression and/or activity of SRPK1 is in diseased tissue or diseased cells.

3. The method according to any of the preceding claims, wherein the disease is a cancer.

4. The method according to any of the preceding claims, wherein the cancer is selected from the group consisting of colon cancer, breast cancer, prostate cancer, pancreatic cancer, brain cancer, ovarian cancer skin cancer, gastrointestinal cancer and lung cancer.

5. The method according to any of the preceding claims, wherein the cancer is resistant to an anti-cancer agent, such as a chemotherapeutic agent.

6. The method according to claim 5, wherein resistance is de novo resistance.

7. The method according to claim 5, wherein resistance is acquired resistance.

8. The method according to any of the preceding claims, wherein the composition comprises a further medicament.

9. The method according to any of the preceding claims, wherein the composition is co-administered with a further medicament.

10. The method according to any of the preceding claims, wherein the composition is administered before, during and/or after the subject has received treatment with a further medicament, optionally wherein the treatment by the further medicament has not been effective.

11. The method according to any of the preceding claims, wherein the further medicament is an anti-cancer agent.

12. The method according to any of the preceding claims, wherein the further medicament is a chemotherapeutic agent selected from the group consisting of topoisomerase inhibitors, anti-hormone agents, alkylating agents, mitotic inhibitors, antimetabolites, anti-tumour antibiotics, corticosteroids, targeted anti-cancer agents, differentiating agents and immunotherapy.

13. The method according to any of the preceding claims, wherein the chemotherapeutic agent is an anti-hormonal agent, such as an anti-estrogen for example tamoxifen, an aromatase inhibitor, a selective estrogen receptor modulator (SERM) such as Fulvestrant, or an anti-progestogen.

14. The method according to any of the preceding claims, wherein the chemotherapeutic agent is a substrate for a drug efflux pump.

15. The method according to any of the preceding claims, wherein the drug efflux pump is selected from the group consisting of P-glycoprotein (P-gp/ABCB1), multidrug resistance-associated protein 2 (MRP2/ABCC2), and breast cancer resistance protein (BCRP/ABCG2).

16. The method according to claim 14-15, wherein said substrate is a topoisomerase I inhibitor, such as a topoisomerase I inhibitor selected from the group topotecan, irinotecan (CPT-11) and SN-38.

17. The method according to claim 14-15, wherein said substrate is a topoisomerase II inhibitor, such as an anthracycline.

18. The method according to claim 14-15, wherein said substrate is a taxane, such as paclitaxel, docetaxel and abraxane.

19. The method according to any of the preceding claims, wherein the elevated expression and/or activity of SRPK1 in diseased tissue or diseased cells is above 1.2, quantified relative to the expression level and/or activity of SRPK1 in non-diseased tissue or non-diseased cells, wherein the expression level of SRPK1 in the non-diseased tissue or non-diseased cells is set to 1.

20. The method according to claim 19, wherein the elevated expression and/or activity of SRPK1 is at least 1.8, such as at least 2.0, such as at least 2.5, such as at least 3.0, such as at least 3.5, such as at least 4.5, such as at least 5.5, such as at least 6.5, such as at least 7.5, such as at least 8.5, such as at least 9.5, such as at least 10.

21. The method according to any of the preceding claims, wherein the treatment is additive.

22. The method according to any of the preceding claims, wherein the treatment is synergistic.

23. The method according to any of the preceding claims, wherein the SRPK1 inhibitor has an IC50 against SRPK1 of 10 μM or less, such as 8 μM or less, for example 6 μM or less; such as 5 μM or less, for example 4 μM or less, such as 3 μM or less, for example 1 μM or less, such as 0.5 μM or less, for example 0.1 μM or less, such as 10 nM or less, for example 5 nM or less, preferably wherein the IC50 is 5 μM or less.

24. The method according to any of the preceding claims, wherein the SRPK1 inhibitor is of formula (II) 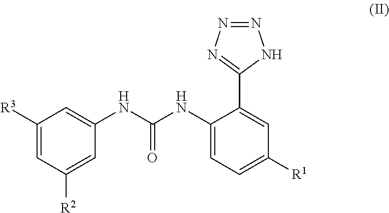
or a pharmaceutically acceptable salt thereof, wherein R¹, R² and R³ are as defined for formula (I).

25. The method according to any of the preceding claims, wherein R¹ represents halo.

26. The method according to any of the preceding claims, wherein R² and R³ independently of each other represent halo or trifluoromethyl.

27. The method according to any of the preceding claims, wherein the SRPK1 inhibitor is selected from:
N-4-Nitrophenyl-N′-[4-bromo-2-(1-H-tetrazol-5-yl)phenyl] urea;
N-3,5-Di(trifluoromethyl)phenyl-N′-[4-bromo-2-(1-H-tetrazol-5-yl)phenyl] urea;
N-3-Trifluoromethylphenyl-N′-[4-(3-nitrophenyl)-2-(1-H-tetrazol-5-yl)phenyl]urea;
N-3-Trifluoromethylphenyl-N′-[4-(4-anilinocarbonylphenyl)-2-(1-H-tetrazol-5-yl)phenyl] urea;
N-3-Trifluoromethylphenyl-N′-[4-(4-trifluoromethylphenyl)-2-(1-H-tetrazol-5-yl)phenyl] urea;
N-(3-Trifluoromethyl-phenyl)-N′-[2-(1-H-tetrazol-5-yl)-phenyl] urea;
N-(3-Trifluoromethyl-phenyl)-N′-[4-bromo-2-(1-H-tetrazol-5-yl)-phenyl] urea;
N-(3-Trilfuoromethyl-phenyl)-N′-[4-phenyl-2-(1-H-tetrazol-5-yl)-phenyl] urea;
N-(3-Chloro-phenyl)-N′-[2-(1-H-tetrazol-5-yl)-phenyl] urea;
N-(3-Trifluoromethyl-phenyl)-N′-[4-amino-2-(1-H-tetrazol-5-yl)-phenyl] urea;
N-(3-Trifluoromethyl-phenyl)-N′-[4-acetylamino-2-(1-H-tetrazol-5-yl)-phenyl]urea;
N-(3-Trifuoromethyl-phenyl)-N′-[4-carbamoyl-2-(1-H-tetrazol-5-yl)-phenyl] urea;
N-(3-Trifluoromethyl-phenyl)-N′-[4-(N″,N″-dimethylcarbamoyl)-2-(1-H-tetrazol-5-yl)-phenyl] urea;
3′-(1-H-tetrazol-5-yl)-4′-[3-(3-trifluoromethyl-phenyl)-ureido]-biphenyl-4-carboxylic acid;
N-(Biphenyl-4-yl)-N′-[2-(1-H-tetrazol-5-yl)-phenyl] urea;
N-(Biphenyl-3-yl)-N′-[2-(1-H-tetrazol-5-yl)-phenyl] urea;
N-(3-Acetyl-phenyl)-N′-[2-(1-H-tetrazol-5-yl)-phenyl] urea;
N-(Biphenyl-3-yl)-N′-[2-(1-H-tetrazol-5-yl)-phenyl] urea;
N-[3-(Pyridin-3-yl)-phenyl]-N′-[2-(1-H-tetrazol-5-yl)-phenyl] urea;
N-(3-Bromo-phenyl)-N′-[4′-(4-methyl-piperazine-1-carbonyl)-3-(1-H-tetrazol-5-yl)-biphenyl-4-yl] urea;
N-(3,5-Dichlorophenyl)-N′-[4-bromo-2-(1-H-tetrazol-5-yl)-phenyl] urea;
N-(3,4-Dichlorophenyl)-N′-[4-bromo-2-(1-H-tetrazol-5-yl)-phenyl] urea;
N-(2-Trifluoromethyl-phenyl)-N′-[4-bromo-2-(1-H-tetrazol-5-yl)-phenyl] urea;
N-(2-Fluorophenyl)-N′-[4-bromo-2-(1-H-tetrazol-5-yl)-phenyl] urea; and
N-(2-Ethylphenyl)-N′-[4-bromo-2-(1-H-tetrazol-5-yl)-phenyl] urea;
or a pharmaceutically acceptable salt thereof.

28. The method according to any of the preceding claims, wherein the SRPK1 inhibitor is selected from:
N-4-Nitrophenyl-N′-[4-bromo-2-(1-H-tetrazol-5-yl)phenyl] urea;
N-3,5-Di(trifluoromethyl)phenyl-N′-[4-bromo-2-(1-H-tetrazol-5-yl)phenyl] urea;
N-3-Trifluoromethylphenyl-N′-[4-(3-nitrophenyl)-2-(1-H-tetrazol-5-yl)phenyl]urea;
N-3-Trifluoromethylphenyl-N′-[4-(4-anilinocarbonylphenyl)-2-(1-H-tetrazol-5-yl)phenyl] urea; and
N-3-Trifluoromethylphenyl-N′-[4-(4-trifluoromethylphenyl)-2-(1-H-tetrazol-5-yl)phenyl] urea;
or a pharmaceutically acceptable salt thereof.

29. The method according to any of the preceding claims, wherein the SRPK1 inhibitor is of formula (III) 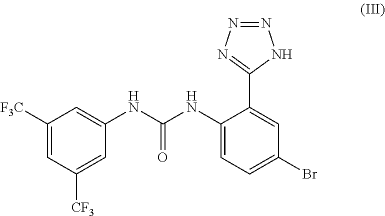 [SMILES: O=C(Nc1cc(C(F)(F)F)cc(C(F)(F)F)c1)Nc1ccc(Br)cc1-c1nnn[nH]1]
or a pharmaceutically acceptable salt thereof.

30. The method according to any of the preceding claims, wherein the SRPK1 inhibitor is of formula (III), the further medicament is irinotecan or SN38 and the disease is colorectal cancer.

31. The method according to any of the preceding claims, wherein the SRPK1 inhibitor is of formula (III), the further medicament is irinotecan or SN38 and the disease is resistant colorectal cancer.

32. The method according to any of the preceding claims, wherein the composition is in the form of tablets or capsules for oral administration.

33. The method according to any of the preceding claims, wherein the composition is in the form of a liquid for intravenous administration or continuous infusion.

34. The method according to any of the preceding claims, wherein the composition is administered topically.

35. The method according to any of the preceding claims, wherein the chemotherapeutic agent is not a metal-based anticancer drug, such as a platinum, ruthenium, gold or titanium-based anticancer drug.

36. The method according to any of the preceding claims, wherein the chemotherapeutic agent is not a platinum-based anticancer drug, such as cisplatin, carboplatin, oxaliplatin or nedaplatin.

37. A method of selecting a subject for treatment according to the method of any of the preceding claims, said method comprising:
a. providing a sample comprising diseased tissue or diseased cells from the subject,
b. determining the expression level and/or activity of SRPK1 in said sample,
c. comparing said expression level and/or activity of SRPK1 with the expression level and/or activity of SRPK1 in a control sample,
wherein an expression level and/or activity of SRPK1 in the sample above the expression level and/or activity in the control sample indicates that the subject is responsive to treatment with the SRPK1 inhibitor as defined in any of the preceding claims.

38. The method according to claim 37, further comprising: determining the expression level and/or activity of BCRP in said sample, and comparing said expression level and/or activity of BCRP with the expression level and/or activity of BCRP in a control sample, wherein an expression level and/or activity of BCRP above the expression level and/or activity of BCRP in the control sample indicates that the subject is responsive to treatment with the SRPK1 inhibitor as defined in any one of the preceding claims.

39. The method according to any of claims 37-38, wherein the sample is a biopsy sample or a tissue resectate.

40. The method according to any of claims 37-38, wherein the sample is a body fluid sample comprising diseased cells, optionally wherein the sample is a blood sample or a spinal fluid sample.

41. The method according to any of claims 37-40, wherein the control sample is obtained from the same subject as the sample comprising diseased tissue or diseased cells and is a sample comprising healthy tissue or healthy cells of the same origin as the diseased tissue or diseased cells.

42. The method according to any of claims 37-41, wherein the control sample is obtained from one or more healthy subjects and comprises healthy tissue or healthy cells of the same origin as the diseased tissue or diseased cells.

43. The method according to any of claims 37-42, wherein the diseased tissue is cancerous tissue.

44. The method according to any of claims 37-42, wherein the diseased cells are cancer cells such as circulating tumor cells.

45. The method according to any of claims 37-43, wherein the expression level of SRPK1 is measured at the mRNA or protein level.

46. The method according to any of claims 37-45, further comprising administering the composition comprising the SRPK1 inhibitor as defined in any of the preceding claims to the subject.

47. A method for treatment of a disease characterized by an elevated expression and/or activity of Serine Arginine Protein Kinase 1 (SRPK1) comprising administering to a subject an effective amount of a composition comprising the SRPK1 inhibitor as defined in any of the preceding claims and optionally a further medicament, wherein a sample comprising diseased tissue or diseased cells obtained from said subject comprises an elevated expression and/or activity of SPRK1.

48. A composition comprising an effective amount of a SRPK1 inhibitor as defined in any of the preceding claims for use in the treatment of a disease characterized by an elevated expression and/or activity of Serine Arginine Protein Kinase 1 (SRPK1).

49. The method according to any of the preceding claims or the composition for use according to claim 48, wherein the composition potentiates the therapeutic effect of a further medicament.

50. Use of a SRPK1 inhibitor as defined in any of the preceding claims for the manufacture of a medicament for treatment of a disease characterized by an elevated expression and/or activity of Serine Arginine Protein Kinase 1 (SRPK1).
